(19) United States
(12) Patent Application Publication
(10) Pub. No.: US 2013/0060036 A1
(43) Pub. Date: Mar. 7, 2013

(54) PROCESS FOR PRODUCTION OF QUINUCLIDINE COMPOUNDS

(71) Applicants: Yutaka Kitagawa, Hiratsuka-shi (JP); Masao Fujita, Takaoka-shi (JP); Kumiko Otaya, Takaoka-shi (JP)

(72) Inventors: Yutaka Kitagawa, Hiratsuka-shi (JP); Masao Fujita, Takaoka-shi (JP); Kumiko Otaya, Takaoka-shi (JP)

(73) Assignee: DAIICHI SANKYO COMPANY, LIMITED, Tokyo (JP)

(21) Appl. No.: 13/503,382

(22) PCT Filed: Oct. 21, 2010
(86) PCT No.: PCT/JP10/68546

§ 371 Date: May 9, 2012

(30) Foreign Application Priority Data
Oct. 23, 2009 (JP) 2009-243947

(51) Int. Cl. C07D 497/20

(52) U.S. Cl. 546/18

(57) ABSTRACT

Provided is a production method for a cis-QMF, which has a low environmental burden and is industrially advantageous. Specifically provided is a production method for a cis-type 2-alkylspiro(1,3-oxathiolane-5,3′)quinuclidine hydrochloride, including: reacting a cis-trans isomer mixture of 2-alkylspiro(1,3-oxathiolane-5,3′)quinuclidine with p-nitrobenzoic acid; resolving the resultant product to produce a cis-type 2-alkylspiro(1,3-oxathiolane-5,3′)quinuclidine p-nitrobenzoate; and converting the p-nitrobenzoate into a hydrochloride.

### FIELD OF THE INVENTION

The present invention relates to a production method for a stereoisomer of 2-alkylspiro(1,3-oxathiolane-5,3′)quinuclidine as typified by cevimeline useful as a therapeutic agent for Sjogren' s syndrome or the like.

### BACKGROUND OF THE INVENTION

2-Alkylspiro(1,3-oxathiolane-5,3′)quinuclidine (hereinafter, referred to as QMF) is an excellent cholinergic agent, and in particular, a cis-type 2-alkylspiro(1,3-oxathiolane-5,3′) quinuclidine (hereinafter, referred to as cis-QMF) has a salivating effect and is widely used as a remedy for mouth dryness symptom of a patient suffering from Sjogren's syndrome (Patent Document 1).

As a production method for the cis-QMF, it has been known that the cis-QMF can be produced by reacting 3-hydroxy-3-mercaptomethylquinuclidine (hereinafter, referred to as QHT) with an aldehyde in the presence of a boron trifluoride-ether complex to produce a cis-trans mixture of QMF and performing a fractional crystallization method or the like (Patent Document 1). Further, there have also been known methods each including subjecting a trans-type QMF (hereinafter, referred to as trans-QMF) separated by the fractional crystallization method to an action of a metal halide, sulfuric acid, or an organic sulfonic acid to isomerize the trans-QMF into the cis-QMF (Patent Documents 2, 3, and 4).

Further, there have been reported: a method including reacting QHT with an aldehyde in the presence of a catalyst selected from the group consisting of a tin halide, a phosphorus oxo acid, an oxyhalide, and an organic sulfonic acid to produce the cis-QMF; and a method including isomerizing the trans-QMF into the cis-QMF in the presence of a tin halide (Patent Document 5). In addition, there has also been reported a method including reacting a cis-trans mixture of QMF with an organic sulfonic acid such as camphorsulfonic acid to produce the cis-QMF (Patent Document 6).

### PRIOR ART DOCUMENT

### Patent Document

[Patent Document 1] JP-A-1986-280497
[Patent Document 2] JP-A-1989-16787
[Patent Document 3] JP-A-1989-45387
[Patent Document 4] JP-A-1989-104079
[Patent Document 5] JP-A-1996-319287
[Patent Document 6] US 2009/0182146

### SUMMARY OF THE INVENTION

However, each of the conventional production methods performs a reaction in an organic solvent, has a high environmental burden, and requires a large amount of energy for recovering the organic solvent. In addition, in each of the conventional methods, a metal halogen reagent is used, but the metal halogen reagent is unfavorable for industrial applications because the reagent is easily deactivated by moisture or water or the like. Therefore, a method performed using no metal halogen reagent has been required. Further, the methods provide insufficient reaction yields and have been required to be further improved.

Therefore, the present invention is to provide a production method for the cis-QMF, which has a low environmental burden and is industrially advantageous.

In view of the foregoing, the present inventors have made various studied various production steps from QHT to a cis-QMF in an aqueous solvent. As a result, they have found that a cis-trans mixture of QMF can be obtained efficiently by reacting QHT with an aldehyde in an aqueous solvent in the presence of an acid catalyst which is safe and industrially and easily available. They have also found that a cis-QMF can be easily separated by reacting the resultant cis-trans QMF mixture with p-nitrobenzoic acid to resolve the resultant mixture and that a trans-QMF separated in a filtrate can be efficiently isomerized into the cis-trans mixture of QMF. Based on the foregoing findings, they have completed the present invention.

The present invention provides the following invention.

(1) A production method for a cis-type 2-alkylspiro(1,3-oxathiolane-5,3′)quinuclidine hydrochloride, including reacting a cis-trans isomer mixture of 2-alkylspiro(1,3-oxathiolane-5,3′)quinuclidine with p-nitrobenzoic acid, resolving the resultant product to produce a cis-type 2-alkylspiro(1,3-oxathiolane-5,3′)quinuclidine p-nitrobenzoate, and converting the p-nitrobenzoate into a hydrochloride.
(2) The production method according to the above-mentioned item (1), in which the cis-trans isomer mixture of 2-alkylspiro(1,3-oxathiolane-5,3′)quinuclidine is reacted with p-nitrobenzoic acid to produce a cis-trans isomer mixture of 2-alkylspiro(1,3-oxathiolane-5,3′)quinuclidine p-nitrobenzoate and the resultant product is resolved to produce the cis-type 2-alkylspiro(1,3-oxathiolane-5,3′)quinuclidine p-nitrobenzoate.
(3) The production method according to the above-mentioned item (1), in which a sulfuric acid aqueous solution of the cis-trans isomer mixture of 2-alkylspiro(1,3-oxathiolane-5,3′)quinuclidine is reacted with p-nitrobenzoic acid and sodium hydroxide to crystallize the cis-type 2-alkylspiro(1,3-oxathiolane-5,3′)quinuclidine p-nitrobenzoate.
(4) The production method according to any one of the above-mentioned items (1) to (3), in which the cis-trans isomer mixture of 2-alkylspiro(1,3-oxathiolane-5, 3) quinuclidine includes a product obtained by reacting 3-hydroxy-3-mercaptomethylquinuclidine with an aldehyde in an aqueous solvent in the presence of an acid catalyst.
(5) The production method according to any one of the above-mentioned items (1) to (4), further including: providing a trans-type 2-alkylspiro(1,3-oxathiolane-5,3′)quinuclidine by the resolution; isomerizing the resultant product to prepare a cis-trans mixture of 2-alkylspiro(1,3-oxathiolane-5,3′)quinuclidine; and using the mixture as a raw material.
(6) The production method according to the above-mentioned item (5), in which the isomerization reaction is performed by reacting the trans-type 2-alkylspiro(1,3-oxathiolane-5,3′)quinuclidine with (a) a boron trifluoride-ether complex and p-nitrobenzoic acid or (b) hydrochloric acid or hydrobromic acid and an aldehyde, in an organic solvent.
(7) The production method according to any one of the above-mentioned items (1) to (6), in which the cis-trans isomer mixture of 2-alkylspiro(1,3-oxathiolane-5,3′)quinuclidine is used as an organic solvent solution or a sulfuric acid aqueous solution.
(8) A production method for a cis-trans isomer mixture of 2-alkylspiro(1,3-oxathiolane-5,3′)quinuclidine, including reacting 3-hydroxy-3-mercaptomethylquinuclidine with an aldehyde in an aqueous solvent in the presence of an acid catalyst.
(9) A production method for a cis-trans mixture of 2-alkylspiro(1,3-oxathiolane-5,3′)quinuclidine, including reacting a trans-type 2-alkylspiro(1,3-oxathiolane-5,3′)quinuclidine with (a) a boron trifluoride-ether complex and p-nitrobenzoic acid or (b) hydrochloric acid or hydrobromic acid and an aldehyde, in an organic solvent.
(10) A cis-type 2-alkylspiro(1,3-oxathiolane-5,3′)quinuclidine p-nitrobenzoate.
(11) A cis-type 2-methylspiro(1,3-oxathiolane-5,3′)quinuclidine p-nitrobenzoate.

The cis-trans mixture of QMF obtained through a reaction in an aqueous solvent according to the present invention can be reutilized in the resolution. This method is a resolution method, and hence a procedure for isomerizing a trans-QMF in a filtrate to perform efficient recovery and reutilization (resolution) as a cis-trans mixture of QMF is an important process. As conventional methods, isomerization methods each using a metal halogen, sulfuric acid, or an organic sulfonic acid have been reported (Patent Documents 2, 3, and 4), but both of the reaction yields and isomerization rates is insufficient.

The present invention includes the step of providing the cis-trans mixture of QMF from QHT, the resolution step using p-nitrobenzoic acid, and the step of isomerizing the trans-QMF resolved in a filtrate into a cis-trans QMF mixture for reutilization. The above-mentioned series of steps proceeds with high yields in an aqueous solvent system, and hence can produce the cis-QMF in an environmentally friendly and industrially advantageous manner.

### DETAILED DESCRIPTION OF THE INVENTION

A production method of the present invention is represented by the following reaction formula.

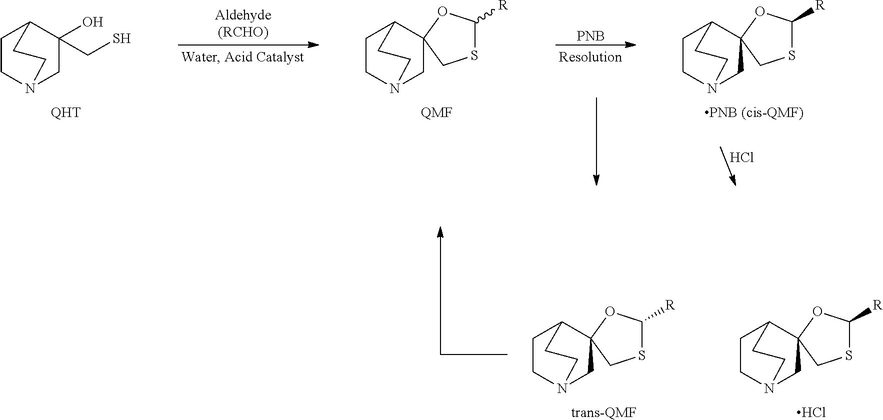

(In the formula, R represents an alkyl group, and PNB represents p-nitrobenzoic acid.)

Hereinafter, each step is described.

(1) Acetalization Step

This step is a step of reacting QHT with an aldehyde in an aqueous solvent in the presence of an acid catalyst to produce a cis-trans isomer mixture of QMF.

Examples of the aldehyde (RCHO) to be used in this reaction include aldehydes each having 2 to 6 carbon atoms such as acetaldehyde, paraldehyde, propylaldehyde, butylaldehyde, and acetaldehyde diethyl acetal or the like. Of these, acetaldehyde and paraldehyde are more preferred. Therefore, examples of R include alkyl groups each having 1 to 5 carbon atoms, and of these, a methyl group is preferred.

Examples of the acid catalyst to be used include hydrobromic acid, sulfuric acid, hydrochloric acid, hydrogen chloride, and perchloric acid or the like. Of these, hydrobromic acid, sulfuric acid, and hydrochloric acid are preferred.

An amount of the aldehyde to be used is preferably 0.5 to 5 equivalent with respect to QHT, and an amount of the acid catalyst to be used is preferably 3 to 7.5 equivalent with respect to QHT. Further, the present invention can be performed in an aqueous solvent and has a low environmental burden. An amount of water to be used has only to be one required for dissolving QHT, and for example, 1 part by weight of water is sufficient with respect to 1 part by weight of QHT. The reaction proceeds under a mild condition of 0 to 40° C., more preferably 20 to 25° C. A reaction time of 5 to 10 hours suffices in ordinary cases.

(2) Resolution Step

This step is a step including reacting a cis-trans isomer of QMF mixture with p-nitrobenzoic acid and resolving the resultant product into the cis isomer and the trans isomer to produce a cis-QMF.p-nitrobenzoate (cis-QMF.PNB). According to this step, a cis-QMF can be resolved efficiently from the cis-trans isomer mixture of QMF by using p-nitrobenzoic acid.

An embodiment of this step includes a method (2-a) involving reacting a cis-trans isomer mixture of QMF with p-nitrobenzoic acid to produce a cis-trans mixture of QMF.p-nitrobenzoate and resolving the resultant mixture into the cis isomer and the trans isomer by a fractional crystallization method or the like to produce a cis-QMF.p-nitrobenzoic acid. In addition, another embodiment thereof includes a method (2-b) involving reacting a sulfuric acid aqueous solution of cis-trans isomer mixture of QMF with p-nitrobenzoic acid and sodium hydroxide to selectively crystallize a cis-QMF.p-nitrobenzoate. The latter embodiment is more preferred because a reaction can be performed in a water-based solvent, and subsequently to the acetalization step performed in an aqueous solvent.

First, the embodiment (2-a) is described. The reaction of the cis-trans isomer mixture of QMF with p-nitrobenzoic acid is performed by reacting 1 to 2 equivalent, preferably 0.9 to 1.5 equivalent of p-nitrobenzoic acid with respect to the cis-trans mixture of QMF in a hydrocarbon-based solvent such as toluene, hexane, or heptane. The reaction temperature is 0 to 70° C., more preferably 20 to 30° C. The resultant cis-trans mixture of QMF.p-nitrobenzoate can be isolated as a crystal. The isolation of the resultant cis-trans mixture of QMF.p-nitrobenzoate can be performed by a usual fractional crystallization method, for example, by dissolving the mixture in water and preferentially crystallizing a cis-QMF.p-nitrobenzoate. During this, a seed crystal of the cis-QMF.p-nitrobenzoate may be added, if necessary. Specifically, the method may be performed by adding water to dissolve the mixture and cooling the resultant slowly. The precipitated crystal can be isolated by filtration, washing with water, drying or the like.

In the embodiment (2-b), specifically, the cis-trans isomer mixture of QMF is dissolved in a sulfuric acid aqueous solution, and p-nitrobenzoic acid is added thereto while adding sodium hydroxide, to thereby selectively crystallize a cis-QMF.p-nitrobenzoate. The amount of sulfuric acid to be used is preferably 0.1 to 2 equivalent, more preferably 0.5 to 1 equivalent with respect to the cis-trans mixture of QMF. The amount of sodium hydroxide to be used is preferably 0.2 to 4 equivalent, more preferably 1 to 2 equivalent with respect to the amount of sulfuric acid added. The amount of p-nitrobenzoic acid to be used is preferably 0.1 to 1 equivalent, more preferably 0.4 to 0.7 equivalent with respect to the cis-trans mixture of QMF.

The cis-QMF.p-nitrobenzoate is selectively crystallized by adding the raw materials, dissolving all the materials by heating, maturing the mixture, and cooling the resultant product slowly. A seed crystal of the cis-QMF.p-nitrobenzoate may be added at around a dissolution temperature. The precipitated crystal can be isolated by filtration, washing with water, drying or the like.

(3) Hydrochlorination Step

This step is a step of converting the cis-QMF.p-nitrobenzoate into a cis-QMF hydrochloride. This reaction may be performed by subjecting the cis-QMF.p-nitrobenzoate to an alkali treatment and after that reacting the resultant product with hydrochloric acid, hydrogen chloride or the like. The alkali treatment may be performed by, for example, adding sodium hydroxide or sodium hydrogen carbonate or the like in an amount of 1 equivalent or more with respect to the cis-QMF.p-nitrobenzoate. Subsequently, hydrochloric acid/an alcohol may be added to precipitate a cis-QMF hydrochloride. In addition, the cis-QMF hydrochloride may be converted into a hydrate such as a cis-QMF hydrochloride 1/2-hydrate, by adjusting the water content.

(4) Isomerization Step

This step is a step of isomerizing a trans-QMF, which is a residue of the cis-QMF.p-nitrobenzoate separated in the resolution step to prepare a cis-trans mixture of QMF. The isomerization step is performed by reacting the trans-QMF, in an organic solvent, with (a) a boron trifluoride-ether complex and p-nitrobenzoic acid or (b) hydrochloric acid or hydrobromic acid and an aldehyde. The trans-QMF used as a raw material of the isomerization step may be obtained by an extraction with an organic solvent such as toluene or xylene from the residue of resolution of the cis-QMF.p-nitrobenzoate.

Examples of the boron trifluoride-ether complex to be used in the method (a) include a boron trifluoride-diethyl ether complex, a boron trifluoride-dibutyl complex, or a boron trifluoride-tert-butyl methyl ether complex. An amount of the boron trifluoride ether complex to be used is preferably 2 to 4 equivalent, more preferably 3 to 3.5 equivalent with respect to the trans-QMF. An amount of p-nitrobenzoic acid to be used is preferably 0.5 to 2 equivalent, more preferably 1 to 1.5 equivalent with respect to the trans-QMF. The method (a) is performed in the organic solvent such as toluene at 20 to 50° C., more preferably at 30 to 40° C., and a reaction time of 1 to 3 hours suffices.

Examples of the aldehyde to be used in the method (b) include the same aldehydes described in the acetalization step, and the organic solvent to be used may be an organic solvent such as toluene but is preferably a two-phase system of organic solvent-water such as toluene-water. More specifically, a two-phase system of toluene-hydrochloric acid aqueous solution or toluene-hydrobromic acid aqueous solution is preferred.

An amount of the aldehyde to be used is preferably 1 to 5 equivalent, more preferably 2 to 3 equivalent with respect to the trans-QMF. An amount of hydrochloric acid or hydrobromic acid to be used is preferably 3 to 6 equivalent, more preferably 5 to 5.5 equivalent with respect to the trans-QMF. The reaction is performed preferably at 0 to 40° C., more preferably at 10 to 15°, and a reaction time of 15 to 20 hours suffices.

In the present invention, it is preferred that the trans-QMF separated in the resolution step is isomerized, and the resultant product is subjected to the resolution step.

### EXAMPLES

Hereinafter, the present invention is described in more detail by way of Examples.

### Example 1

(1) 10.0 g of QHT and 20 mL of water were added to a 100-mL three-necked flask equipped with a stirrer and a thermometer, and the mixture was cooled to 10 to 15° C. 7.63 g of paraldehyde and 48.6 g of a 48% hydrobromic acid aqueous solution were added dropwise, and the mixture was heated to 40° C. and stirred at the same temperature for 20 hours. The reaction solution was cooled to 25° C., and 42 mL of toluene were added to separate the solution. 42 mL of toluene were added again to the aqueous layer to separate the layer, and the separated aqueous layer was cooled to 10 to 15° C. 33 mL of a 28% sodium hydroxide aqueous solution were added to make the layer strongly alkaline, followed by extraction and separation with 84 mL of toluene. 16.8 mL of water were added to the toluene layer to separate the solution, and 0.84 g of activated carbon was added to the separated toluene layer. The mixture was stirred, and the activated carbon was collected by filtration. The collected activated carbon was washed with 16.8 mL of toluene. 7.19 g of p-nitrobenzoic acid were added to the filtrate, and the mixture was stirred to precipitate a crystal as a p-nitrobenzoate. The crystal was dissolved by heating. The solution was cooled slowly to precipitate a crystal, and 50 mL of hexane were added, followed by stirring at 10 to 15° C. for 2 hours. The precipitated crystal was collected by filtration and washed with 34 mL of hexane, and the collected crystal was dried by heating under reduced pressure, to thereby produce 15.71 g of QMB (a cis- and trans-p-nitrobenzoate isomer mixture). It should be noted that the cis isomer/trans isomer ratio of the resultant mixture was analyzed by liquid chromatography, and as a result, the cis isomer/trans isomer ratio was found to be 57.5/42.5.

(2) 35 mL of water were added to 7.00 g of QMB obtained in (1), and QMB was dissolved by heating. The mixture was cooled slowly, and a seed crystal was added at around the dissolution temperature to precipitate a crystal, followed by stirring at 10 to 15° C. for 2 hours. The precipitated crystal was collected by filtration and washed with 7 mL of water, and the collected crystal was dried by heating under reduced pressure, to thereby produce 3.63 g of QCB (a cis- and trans-p-nitrobenzoate isomer mixture enriched with the cis isomer). It should be noted that the cis isomer/trans isomer ratio of the resultant mixture was analyzed by liquid chromatography, and as a result, the cis isomer/trans isomer ratio was found to be 89.6/10.4.

(3) A reaction was performed in the same way as in (2) above, except that no crystal seed was added. The cis isomer/trans isomer ratio of the resultant mixture was analyzed by liquid chromatography, and as a result, the cis/trans ratio was found to be 86.1/13.9.

### Example 2

(1) 500 g of QHT and 500 mL of water were added to a 10-L four-necked flask equipped with a stirrer and a thermometer, and the mixture was cooled to 10 to 15° C. 381.3 g of paraldehyde and 1,945.6 g of a 48% hydrobromic acid aqueous solution were added dropwise, and the mixture was heated to 20 to 30° C. and stirred at the same temperature for 5 hours. The reaction solution was cooled to 10 to 15° C., and 1,350 mL of a 28% sodium hydroxide aqueous solution were added to make the solution strongly alkaline, followed by extraction and separation with 3,750 mL of toluene. 1,500 mL of water were added to the toluene layer to separate the solution, and 1,040 mL of a 10% sulfuric acid aqueous solution were added to the separated toluene layer. The mixture was stirred and separated.

100 mL of a 10% sulfuric acid aqueous solution were added again to the separated toluene layer, and the mixture was stirred and separated. All the sulfuric acid aqueous layers were combined, to thereby produce a QMF/sulfuric acid aqueous solution (a sulfuric acid aqueous solution of a cis-trans isomer mixture).

(2) 192.3 g of p-nitrobenzoic acid and 157 mL of 28% sodium hydroxide were added to the QMF/sulfuric acid aqueous solution obtained in (1), and the mixture was stirred. A crystal precipitated as a p-nitrobenzoate was dissolved by heating, and the solution was cooled slowly. A seed crystal was added at around the dissolution temperature to precipitate a crystal, followed by stirring at 10 to 15° C. for 2 hours. The precipitated crystal was collected by filtration and washed with 500 mL of water, and the collected crystal was dried by heating under reduced pressure, to thereby produce 372.6 g of QCB (a cis- and trans-p-nitrobenzoate isomer mixture enriched with the cis isomer). It should be noted that the cis isomer/trans isomer ratio of the resultant mixture was analyzed by liquid chromatography, and as a result, the cis/trans ratio was found to be 89.8/10.2.

(3) 1,850 mL of water were added to 370.0 g of QCB obtained in (2), and QCB was dissolved by heating. The mixture was cooled slowly, and a seed crystal was added at around the dissolution temperature to precipitate a crystal, followed by stirring at 10 to 15° C. for 2 hours. The precipitated crystal was collected by filtration and washed with 370 mL of water, and the collected crystal was dried by heating under reduced pressure, to thereby produce 303.6 g of QCB-1 (a cis- and trans-p-nitrobenzoate isomer mixture enriched with the cis isomer). It should be noted that the cis isomer/trans isomer ratio of the resultant mixture was analyzed by liquid chromatography, and as a result, the cis/trans ratio was found to be 98.3/1.7.

### Example 3

(1) 131 mL of a 28% sodium hydroxide aqueous solution were added to 2,099.2 g of the filtrate obtained in Example 2 (2) (cis isomer/trans isomer=22.3/77.7, content: 222.2 g in terms of QMF) to make the filtrate strongly alkaline, followed by extraction twice with 2,043 mL of toluene. 817 mL of water were added to the toluene layer to separate the solution, and 40.9 g of activated carbon were added to the separated toluene layer. The mixture was stirred, and the activated carbon was collected by filtration. The collected activated carbon was washed with 409 mL of toluene, and 186.3 g of p-nitrobenzoic acid were added to the filtrate, followed by stirring. The inside of the reaction system was turned into a nitrogen atmosphere, and 553.9 g of a boron trifluoride diethyl ether complex were added. The mixture was heated to 40° C. and stirred for 1.5 hours. The reaction solution was cooled to 10 to 15° C., and 817 mL of water and 1,021 mL of a 28% sodium hydroxide aqueous solution were added to make the solution strongly alkaline. The precipitated insoluble matter was collected by filtration, and the residue was washed with 817 mL of toluene. The filtrate was separated, and the toluene layer was washed with 817 mL of water. Then, 39.5 g of activated carbon were added to the toluene layer, and the mixture was stirred. After filtration, the collected activated carbon was washed with 395 mL of toluene. 513 mL of a 10% sulfuric acid aqueous solution were added to the filtrate, and the mixture was stirred and separated. 79 mL of a 10% sulfuric acid aqueous solution were added again to the separated toluene layer, and the mixture was stirred and separated. All the sulfuric acid aqueous layers were combined, to thereby quantitatively produce a QMF/sulfuric acid aqueous solution (cis isomer/trans isomer=50.3/49.7).

(2) 1,500 mL of water were added to 300.0 g of QCB-1 obtained in (1), and QCB-1 was dissolved by heating. The mixture was cooled slowly, and a seed crystal was added at around the dissolution temperature to precipitate a crystal, followed by stirring at 10 to 15° C. for 2 hours. The precipitated crystal was collected by filtration and washed with 300 mL of water, and the collected crystal was dried by heating under reduced pressure, to thereby produce 264.0 g of QCB-2 (a cis- and trans-p-nitrobenzoate isomer mixture enriched with the cis isomer). It should be noted that the cis isomer/trans isomer ratio of the resultant mixture was analyzed by liquid chromatography, and as a result, the cis isomer/trans isomer ratio was found to be 99.7/0.3.

### Example 4

14 mL of a 28% sodium hydroxide aqueous solution were added to 213.8 g of the filtrate obtained in Example 2 (2) (cis isomer/trans isomer=24.4/75.6, content: 24.4 g in terms of QMF) to make the filtrate strongly alkaline, followed by extraction with 224 mL of toluene. 45 mL of water were added to the toluene layer to separate the solution, and 2.24 g of activated carbon were added to the separated toluene layer. The mixture was stirred, and the activated carbon was collected by filtration. The collected activated carbon was washed with 45 mL of toluene. The filtrate was cooled to 0 to 10° C., and 47.9 g of paraldehyde and 69.2 g of a 35% hydrochloric acid aqueous solution were added, followed by stirring at the same temperature for 15 hours. 74.5 mL of the 28% sodium hydroxide aqueous solution were added to the reaction solution to make the solution strongly alkaline, and the solution was heated to 20 to 30° C. and separated. The toluene layer was washed with 45 mL of water, and 55.3 mL of a 10% sulfuric acid aqueous solution were added. The mixture was stirred and separated. 5.2 mL of a 10% sulfuric acid aqueous solution were added again to the separated toluene layer, and the mixture was stirred and separated. All the sulfuric acid aqueous layers were combined, to produce a QMF/sulfuric acid aqueous solution (cis isomer/trans isomer=51.2/48.8, content: 22.9 g in terms of QMF).

### Example 5

1,000 mL of water and 66 mL of a 28% sodium hydroxide aqueous solution were added to 200.0 g of the QCB-2 obtained in Example 3 to make the solution strongly alkaline, and extraction was performed four times with 1,000 mL of n-hexane. 200 mL of a 1 mol/L sodium hydroxide aqueous solution were added to the extracted n-hexane layer to separate the solution, and washing was performed with 200 mL of water to separate the solution. 100 g of anhydrous sodium sulfate and 10 g of activated carbon were added to the n-hexane layer, and the mixture was stirred and filtrated. The residue was washed with 800 mL of n-hexane. In a nitrogen atmosphere, the filtrate was cooled to 10 to 15° C., and 284.3 g of a 7% hydrochloric acid/2-propanol solution were added dropwise to precipitate it as a hydrochloride. The mixture was stirred at the same temperature for 2 hours. The precipitated crystal was collected by filtration and washed with 400 mL of a mixed solution of n-hexane/2-propanol (9/1 volume ratio), and the crystal collected by filtration was dried by heating under reduced pressure. The dried crystal was allowed to stand in an atmosphere where the humidity was controlled with a saturated potassium carbonate aqueous solution to hydrate the crystal, thereby providing 117.7 g of cevimeline hydrochloride hydrate.

### Example 6

9.8 g of p-nitrobenzoic acid and 8.3 mL of 28% sodium hydroxide were added to the QMF/sulfuric acid aqueous solution obtained in Example 4, and the mixture was stirred. A crystal precipitated as a p-nitrobenzoate was dissolved by heating, and the solution was cooled slowly. A seed crystal was added at around the dissolution temperature to precipitate a crystal, followed by stirring at 10 to 15° C. for 2 hours. The precipitated crystal was collected by filtration and washed with 22.4 mL of water, and the collected crystal was dried by heating under reduced pressure, to thereby produce 17.1 g of QCB (a cis- and trans-p-nitrobenzoate isomer mixture enriched with the cis isomer). It should be noted that the cis isomer/trans isomer ratio of the resultant mixture was analyzed by liquid chromatography, and as a result, the cis isomer/trans isomer ratio was found to be 88.5/11.5.

1. A method for producing a cis-type 2-alkylspiro(1,3-oxathiolane-5,3′)quinuclidine hydrochloride, the method comprising:
(I) reacting a cis-trans isomer mixture of 2-alkylspiro(1,3-oxathiolane-5,3′)quinuclidine with p-nitrobenzoic acid, to obtain an intermediate product;
(II) resolving the intermediate product to produce a cis-type 2-alkylspiro(1,3-oxathiolane-5,3′)quinuclidine p-nitrobenzoate; and
(III) converting the p-nitrobenzoate into a hydrochloride.

2. The method of claim 1, wherein the intermediate product is a cis-trans isomer mixture of 2-alkylspiro(1,3-oxathiolane-5,3′)quinuclidine p-nitrobenzoate.

3. The method of claim 1, wherein a sulfuric acid aqueous solution of the cis-trans isomer mixture of 2-alkylspiro(1,3-oxathiolane-5,3′)quinuclidine is reacted with p-nitrobenzoic acid and sodium hydroxide to crystallize the cis-type 2-alkylspiro(1,3-oxathiolane-5,3′)quinuclidine p-nitrobenzoate.

4. The method of claim 1, wherein the cis-trans isomer mixture of 2-alkylspiro(1,3-oxathiolane-5,3′)quinuclidine comprises a product obtained by reacting 3-hydroxy-3-mercaptomethylquinuclidine with an aldehyde in an aqueous solvent in the presence of an acid catalyst.

5. The method of claim 4, wherein the acid catalyst comprises hydrobromic acid, hydrochloric acid, sulfuric acid, or perchloric acid.

6. The method of claim 4, wherein the acid catalyst comprises hydrobromic acid.

7. The method according to of claim 1, further comprising:
resolving the intermediate product to produce a trans-type 2-alkylspiro(1,3-oxathiolane-5,3′)quinuclidine; and
isomerizing the trans-type 2-alkylspiro(1,3-oxathiolane-5,3′, to prepare a cis-trans mixture of 2-alkylspiro (1,3-oxathiolane-5,3′)quinuclidine, and employing the cis-trans mixture in the reacting (I).

8. The method of claim 7, wherein the isomerizing comprises reacting the trans-type 2-alkylspiro(1,3-oxathiolane-5,3′)quinuclidine with (a) a boron trifluoride-ether complex and p-nitrobenzoic acid or (b) hydrochloric acid or hydrobromic acid and an aldehyde, in an organic solvent.

9. The method of claim 1, wherein the cis-trans isomer mixture of 2-alkylspiro(1,3-oxathiolane-5,3′)quinuclidine is in the form of an organic solvent solution or a sulfuric acid aqueous solution.

10. The method of claim 4, wherein the aldehyde comprises acetaldehyde or paraldehyde.

11. A method for producing a cis-trans isomer mixture of 2-alkylspiro(1,3-oxathiolane-5,3′)quinuclidine, the method comprising:
reacting 3-hydroxy-3-mercaptomethylquinuclidine with an aldehyde in an aqueous solvent in the presence of an acid catalyst.

12. The method of claim 11, wherein the acid catalyst comprises hydrobromic acid, hydrochloric acid, sulfuric acid, or perchloric acid.

13. The method of claim 11, wherein the acid catalyst comprises hydrobromic acid.

14. The method of claim 11, wherein the aldehyde comprises acetaldehyde or paraldehyde.

15. A method for producing a cis-trans mixture of 2-alkylspiro(1,3-oxathiolane-5,3′)quinuclidine, the method comprising:
reacting a trans-type 2-alkylspiro(1,3-oxathiolane-5,3′)quinuclidine with (a) a boron trifluoride-ether complex and p-nitrobenzoic acid or (b) hydrochloric acid or hydrobromic acid and an aldehyde, in an organic solvent.

16. A cis-type 2-alkylspiro(1,3-oxathiolane-5,3′)quinuclidine p-nitrobenzoate.

17. A cis-type 2-methylspiro(1,3-oxathiolane-5,3′)quinuclidine p-nitrobenzoate.

18. The method of claim 8, wherein the isomerizing comprises reacting the trans-type 2-alkylspiro(1,3-oxathiolane-5,3′)quinuclidine with (a) a boron trifluoride-ether complex and p-nitrobenzoic acid in an organic solvent.

19. The method of claim 8, wherein the isomerizing comprises reacting the trans-type 2-alkylspiro(1,3-oxathiolane-5,3′)quinuclidine with (b) hydrochloric acid or hydrobromic acid and an aldehyde in an organic solvent.

20. The method of claim 4, wherein the aldehyde comprises paraldehyde.
